(19) United States
(12) Patent Application Publication
(10) Pub. No.: US 2008/0161841 A1
(43) Pub. Date: Jul. 3, 2008

(54) Cutting device and method of vessel harvesting

(76) Inventors: Cynthia T. Clague, Minnetonka, MN (US); Michael J. Hobday, Lino Lakes, MN (US); Raymond W. Usher, Coon Rapids, MN (US); Roderick E. Briscoe, Rogers, MN (US); Katherine S. Olig, Eden Prairie, MN (US); Ana R. Menk, Minneapolis, MN (US); Christopher P. Olig, Eden Prairie, MN (US); Eric A. Meyer, Andover, MN (US); Steven C. Christian, New Brighton, MN (US); Thomas P. Daigle, Corcoran, MN (US); Robert H. Reetz, Rackford, MN (US); Jeffrey D. Sandstrom, Forest Lake, MN (US); James R. Keogh, Maplewood, MN (US); Matthew D. Bonner, Plymouth, MN (US); Scott E. Jahns, Hudson, WI (US); Philip J. Haarstad, Chanhassen, MN (US)

Correspondence Address: MEDTRONIC, INC., 710 MEDTRONIC PARKWAY NE, MINNEAPOLIS, MN, 55432-9924, US

(21) Appl. No.: 11/974,914

(22) Filed: Oct. 16, 2007

Related U.S. Application Data
(60) Provisional application No. 60/852,020, filed on Oct. 16, 2006.

(51) Int. Cl. A61B 17/3205

(52) U.S. Cl. 606/159

(57) ABSTRACT

Embodiments of the invention provide a cutting device and method of vessel harvesting. The cutting device can include at least one tubular member, a cutting element, and a centering member. The cutting device can include at least one tubular member with a flexible section and a cutting element. The method of vessel harvesting can include spacing a cutting element of the cutting device from the vessel as the cutting element is advanced over the vessel.

### RELATED APPLICATIONS

This application claims priority under 35 U.S.C. § 119 to U.S. Provisional Patent Application Ser. No. 60/852,020, filed on Oct. 16, 2006, the entire contents of which is hereby incorporated by reference.

### FIELD OF THE INVENTION

This invention relates generally to biomedical systems and methods. More specifically, the invention relates to systems and methods for harvesting a vessel section.

### BACKGROUND

Heart disease, specifically coronary artery disease, is a major cause of death, disability, and healthcare expense in the United States and other industrialized countries. A common form of heart disease is atherosclerosis, in which the vessels leading to the heart are damaged or obstructed by plaques containing cholesterol, lipoid material, lipophages, and other materials. When severely damaged or obstructed, one or more of the vessels can be bypassed during a coronary artery bypass graft (CABG) procedure. CABG surgery is performed about 350,000 times annually in the United States, making it one of the most commonly performed major operations.

To prevent rejection, the graft material is preferably a blood vessel harvested from elsewhere within a patient's body. The most frequently used bypass vessel is the saphenous vein from the leg. Because the venous system of the leg is redundant, other veins that remain within the patient's leg are able to provide return blood flow following removal of the saphenous vein.

Various methods have been used to harvest the saphenous vein. Until recently, the typical procedure involved making a single long incision that overlies the entire length of the vein, extending from a patient's groin to at least the knee and often to the ankle. This method results in substantial postoperative pain, with patients frequently complaining more of discomfort at the site of the leg vein harvesting than of pain from their CABG surgery wound. In addition, such an extensive incision site is subject to infection and delayed healing, especially in patients with poor circulation, which not infrequently accompanies coronary artery disease. The disfiguring scar from such a large incision is also of concern to some patients.

Less invasive procedures are preferred, and surgical devices and techniques now exist that allow the saphenous vein to be harvested through one or more small, transverse incisions along the length of the vein, generally using an endoscope. Endoscopic procedures yield reduced wound complications and superior cosmetic results compared with traditional methods of vein harvesting. However, this procedure requires considerable manipulation of the vein, has a high conversion rate when visualization is obscured by bleeding or the procedure is taking too long and often requires stitches to repair the vein following harvest. Further, it is generally tedious, time consuming, and relatively complex, requiring extensive accessory equipment and a substantial learning curve for the surgeon.

### SUMMARY

Some embodiments of the invention provide a cutting device for use in harvesting a section of a vessel from surrounding tissue. The cutting device can include at least one tubular member adapted to surround the vessel along the section of the vessel to be harvested. The cutting device can include a cutting element coupled to the at least one tubular member. The cutting element can be adapted to be moved along the section of the vessel in order to cut the tissue around the vessel. The cutting device can include a centering member coupled to one of the at least one tubular member and the cutting element. The centering member can be adapted to keep the vessel spaced from the cutting element as the cutting element cuts the tissue around the vessel.

According to one method of the invention, a section of a vessel can be harvested from surrounding tissue by making a first incision at a proximal end of the section of the vessel, and making a second incision at a distal end of the section of the vessel. The method can include introducing a vessel support device into the vessel, and orienting a cutting device coaxially with the vessel support device. The method can also include advancing the cutting device over the vessel to core out the section of the vessel and a portion of the surrounding tissue, and spacing a cutting element of the cutting device from the vessel as the cutting element is advanced over the vessel.

Some embodiments of the invention provide a cutting device including at least one tubular member with a flexible section on a portion of a distal end. The cutting device can include a cutting element coupled adjacent to the flexible section of the at least one tubular member. The cutting element can be adapted to be moved along the section of the vessel in order to cut the tissue around the vessel.

### DESCRIPTION OF THE DRAWINGS

FIG. 1 is an illustration of one embodiment of a cutting device for harvesting a vessel section in accordance with some embodiments of the invention;

FIGS. 2A-2M illustrate various views of components for centering in accordance with some embodiments of the invention;

FIGS. 3A-3B are flow diagrams of methods for harvesting a vessel section in accordance with some embodiments of the invention;

FIG. 4 is an illustration of a distal end of a cutting device for harvesting a vessel section in some embodiments of the invention;

FIG. 5 is an exploded illustration of a distal end of a cutting device for harvesting a vessel section in some embodiments of the invention; and

FIGS. 6A-6J are illustrations of distal ends of a cutting device for harvesting a vessel section in some embodiments of the invention.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

As used in this specification and in the appended claims, the terms “distal” and “proximal” are with reference to the operator when the device is in use.

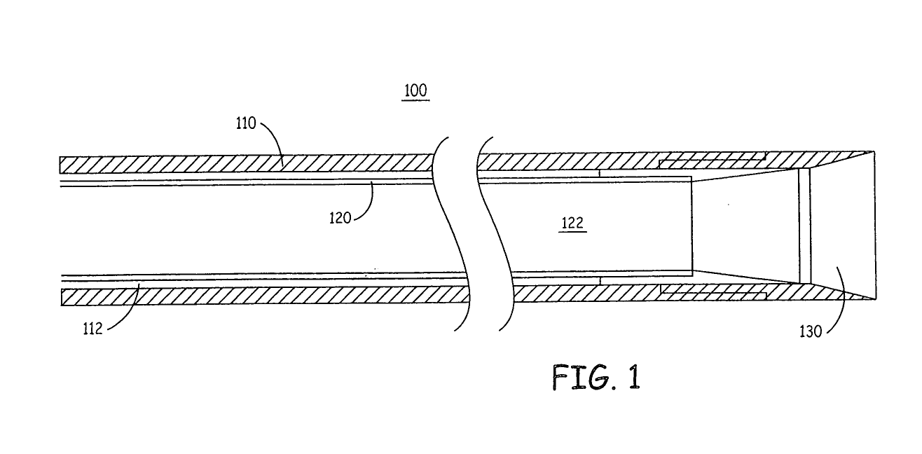

FIG. 1 illustrates a cutting device 100 according to one embodiment of the invention for harvesting a vessel section. The cutting device 100 can include one or more tubular members, such as an outer tubular member 110 and an inner tubular member 120, along with a cutting element 130.

The outer tubular member 110 can be substantially rigid and can be constructed of an appropriate biocompatible material, such as a polymer or stainless steel. In some embodiments, a distal portion of the outer tubular member 110 can be flexible. For example, a section of the outer tubular member 110 proximal to the cutting element 130 can include a bellows-like structure to aid in directing the cutting device 100 over the vessel section to be harvested.

The length of the outer tubular member 110 can be based on the length of the vessel section to be harvested. For example, a length of 30 to 60 centimeters can be appropriate for harvesting a section of a typical saphenous vein. The outer tubular member 110 can be long enough to core out the entire vessel section desired.

The surfaces of the cutting device 100 can be coated with a material to decrease friction between the cutting device 100 and the tissue and between the elements of the cutting device 100. The coating material can be parylene, Teflon, or other slippery, lubricious coatings.

In some embodiments, the cutting element 130 includes one or more blades positioned adjacent to the distal end of the outer tubular member 110. The cutting element 130 can be mounted either inside or outside of the outer tubular member 110. In some embodiments, the diameter of the outer tubular member 110 combined with the positioning of the cutting element 130 determines the diameter of the cored-out vessel and adjoining tissue section that is harvested. In one embodiment, the diameter of the core is adequate to avoid slicing the edges of the vessel being harvested as well as to transect branch vessels, such that the portions of the branch vessels that remain attached to the vessel section are long enough to tie off or otherwise seal to yield a vessel section appropriate for use as a graft, for example in a CABG procedure.

FIG. 1 illustrates that the cutting element 130 can include one or more blades. The number and shape of the blades that form the cutting element 130 can be varied. For example, the outer tubular member 110 can carry one or more of the following: a curved blade, a blade having a taper on an outside surface, a blade having a taper on an inside surface, a blade having a blunt edge on a first surface and a sharp edge on a second surface, a ring having a serrated edge, a ring having a sharpened edge, a ring having an angle, a ring having a beveled or tapered edge, a ring having a scalloped edge, two concentric rings with multiple cutting edges that pass scissor-like by each other, etc. Each different type of cutting element 130 can interact with the tissue surrounding the vessel in a particular way. For example, a blade having a blunt edge on the inside and a sharp edge on the outside can provide a small buffer space between the vessel and the cutting edge of the blade.

In one embodiment, the inner tubular member 120 can be received within a lumen 112 of the outer tubular member 110. The inner tubular member 120 can be somewhat longer than the outer tubular member 110 to allow the two members to be manipulated independently. The lumen 112 of the outer tubular member 110 can provide a close-sliding fit for the inner tubular member 120, allowing the inner tubular member 120 to slide both longitudinally and rotationally within the outer tubular member 110.

In one embodiment, the inner tubular member 120 can be substantially rigid and can be constructed of an appropriate biocompatible material, such as a polymer or stainless steel. In one embodiment, a distal portion of the inner tubular member 120 can include one or more flexible materials. The flexible section of the inner tubular member 120 can be, for example, a soft polymer, a wire-reinforced polymer, a perforated section, a bellows section, or a jointed section.

The lumen 122 of inner tubular member 120 is sized to accommodate the vessel section being harvested and can taper inwardly from the distal end, the inner diameter of a distal portion thus being larger than the inner diameter of a proximal portion. An inward taper can lightly compress the vessel section to provide better centering of the inner tubular member 120 on the vessel section.

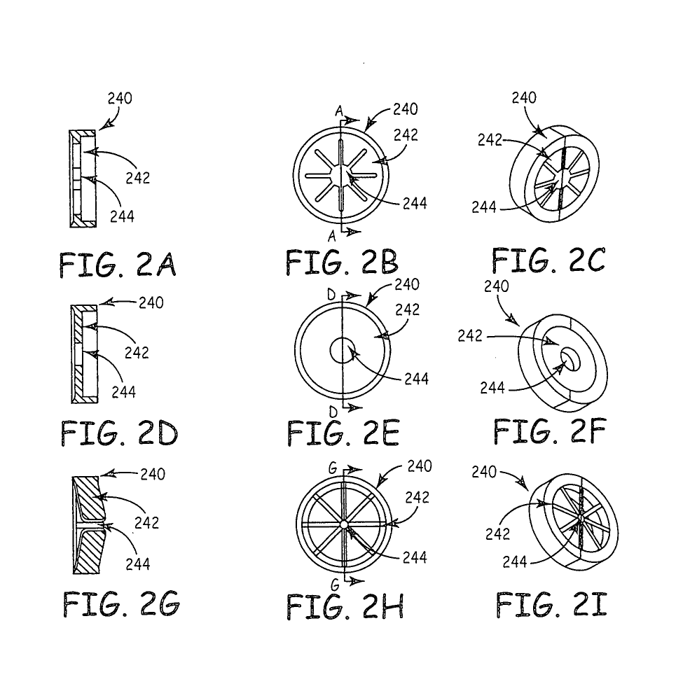

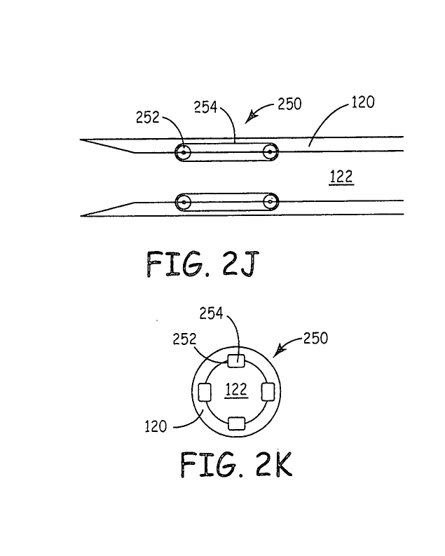

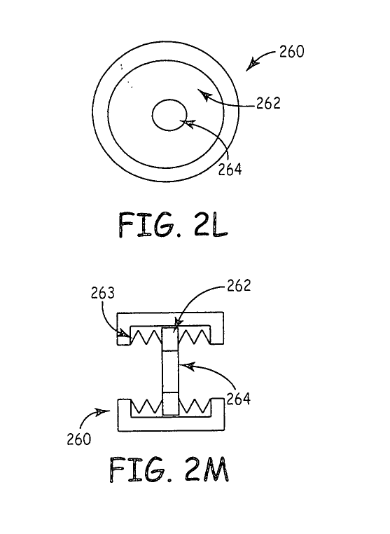

The cutting device 100 can include a component to aid in positioning the device relative to the vessel to provide better centering of the vessel within the device. FIGS. 2A-2M illustrate several examples of centering members according to various embodiments of the invention. The centering member can be positioned within the lumen 112, 122 of either the inner or outer tubular members 110, 120 or within the cutting element 130. The centering member can also be positioned within a lumen of the cutting element 130. Positioning the centering member within the inner tubular member 120 may allow maximum freedom of movement for the inner tubular member 120 within the outer tubular member lumen 112. The inner tubular member 120 can be held substantially stationary with the vessel centered, while the outer tubular member 110 is moved along the vessel section.

FIGS. 2A-2I illustrate three angles of three different embodiments of a centering member 240 that includes centering elements 242 and apertures 244. The centering elements 242 and apertures 244 can assume shapes other than those shown. The centering elements 242 as shown in FIGS. 2A-2C can be flexible and thus capable of bending or being displaced as the cutting device 100 is advanced over the vessel. If the cutting device 100 deviates from a position centered on the vessel, one or more of the centering elements 242 is put under greater tension than the other centering elements 242 and, to reduce the tension, directs the cutting device 100 back into a position centered on the vessel. The centering member 240 and the centering elements 242 can be constructed of one or more biocompatible materials that are both flexible and resilient, for example one or more polymers or rubbers.

In one embodiment, the centering elements 242 can include one or more protrusions that extend into the lumen 112, 122 of the inner or outer tubular member 110, 120. For example, a single ring-like structure, as shown in FIGS. 2D-2F, can extend into the lumen 112, 122 near a proximal end of the inner or outer tubular member 110, 120. In another embodiment, as shown in FIGS. 2G-2I, multiple individual protrusions can be interspaced around the inner wall of the tubular member 110, 120. Both the ring-like structure and the individual protrusions exert a force on the vessel and surrounding tissue, helping to center the vessel within the cutting device 100. The shape and number of protrusions can be varied to achieve maximum centering.

FIGS. 2J and 2K illustrate two views of a centering member 250 that comprises bearings 252 and rollers 254. FIG. 2J is a cross-sectional side view of the inner tubular member 120 showing two such mechanisms. The cross-sectional end view shown in FIG. 2K illustrates four mechanisms interspaced around inner lumen 122.

FIGS. 2L and 2M illustrate two views of a centering member 260 that comprises centering element 262, springs 263, and an aperture 264. FIG. 2M is a cross-sectional side view of the centering member 260. In one embodiment, the centering element 262 can include a rigid plate or disk having an aperture 264. One or more pairs of springs 263 can be attached to the centering element 262 and positioned around the aperture 264. The springs 263 can be provided on either side of the centering element 262 or on both sides of the centering element 262, as shown in FIG. 2M. In one embodiment, one or more pairs of elastic members, e.g., bands, can be substituted for the springs 263. The elastic members can be made of one or more rubbers or resilient-materials.

In another embodiment, the centering member can be a system including at least one sensor for tracking the location of the cutting device 100 relative to a rod, a dilator, a catheter, or a guidewire, for example, positioned within the vessel to be harvested. In this system, at least one Hall-effect sensor, for example, can detect the presence of a metal, for example, in the rod, dilator, catheter, or guidewire, placed within the vessel. Software associated with the sensor(s) can display concentric circles (or other geometrical shapes) representing the positions of the cutting device 100 and the rod, dilator, catheter, or guidewire. In one embodiment, an operator can center the cutting device 100 over the vessel by maintaining the circle representing the rod, dilator, catheter, or guidewire centered within the circle representing the cutting device 100. Alternatively, software associated with the sensor(s) can provide an audible indication of the relative locations of the cutting device 100 and rod, dilator, catheter, or guidewire. For example, the volume or pitch can change if the cutting device 100 deviates off center with respect to the rod, dilator, catheter, or guidewire.

In another embodiment, the centering member includes two magnetic or electromagnetic fields that repel each other. One intravascular field can be located within a catheter or guidewire inside the vessel and the opposing field can be located on the cutting element 130 and/or the outer tubular member 110. The forces can repel each other keeping the cutting element 130 and the outer tubular member 110 away from the inside of the vessel allowing a tissue core to be cut around the vessel without compromising the vessel.

In some embodiments, the outer and inner tubular members 110, 120 can be advanced over a vessel section to core out the vessel section along with tissue adjoining the vessel section. The inner and outer tubular members 110, 120 can be advanced independently. For example, the inner tubular member 120 can be advanced first to hold the vessel and the surrounding tissue while the outer tubular member 110 is advanced second to cut the tissue being held by the inner tubular member 120. Alternately, the outer tubular member 110 can be advanced first to cut the tissue and the inner tubular member 120 can be advanced second to center and hold the tissue. The process of incrementally advancing the tubular members 110, 120 can be repeated until the entire section of vessel has been excised. In one embodiment, advancing the inner tubular member 120 ahead of the outer tubular member 110 can protect the walls of the vessel from the cutting element 130 positioned on the outer tubular member 110. The outer and inner tubular members 110, 120 can also be advanced together with the outer tubular member 110 rotating and the inner tubular member 120 not rotating. Alternative methods of advancing the tubular members 110, 120 include pushing and/or pulling, rotating, and twisting first in one direction and then in the other direction. In one embodiment, the outer tubular member 110 and the inner tubular member 120 can be twisted in opposite directions to provide a scissoring action.

Another embodiment of a cutting device 100 is similar to that described above and illustrated in FIG. 1, with the exception that the cutting device 100 comprises a single tubular member 110 that is advanced over a vessel section to core out the vessel section and tissue adjoining the vessel section. The single tubular member 110 has a cutting element 130 positioned adjacent to its distal end. In one embodiment, a distal portion of the tubular member 110 can extend beyond a distal end of the cutting element 130 to protect the vessel section being harvested from being cut by the cutting elements 130, e.g., blade(s). The blade(s) can assume a variety of forms, including, but not limited to, a ring having a serrated edge, a ring having a sharpened edge, a straight blade, a curved blade, a blade having a taper on an outside surface, a blade having a taper on an inside surface, a blade having a blunt edge on a first surface and a sharp edge on a second surface, a ring having an angle, a ring having a scalloped edge, two concentric rings with multiple cutting edges that pass scissor-like by each other, etc. The centering members 240, 250, 260 as described above can be positioned within the lumen 112 of the single tubular member 110. A distal portion of the tubular member 110 can be flexible to aid in directing the cutting device 100 over the vessel section to be harvested. In some embodiments, the flexible portion can comprise at least a portion of the cutting element 130.

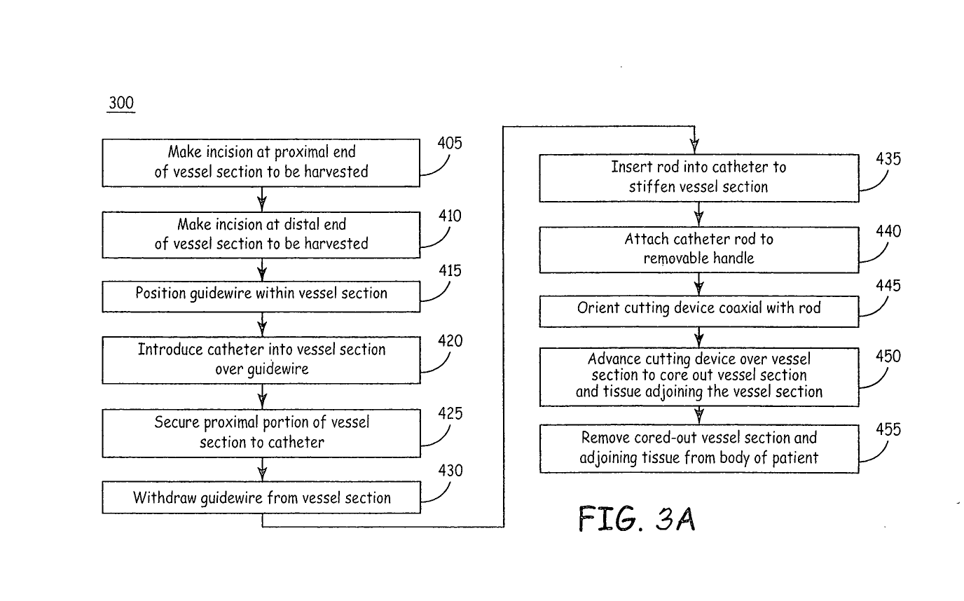

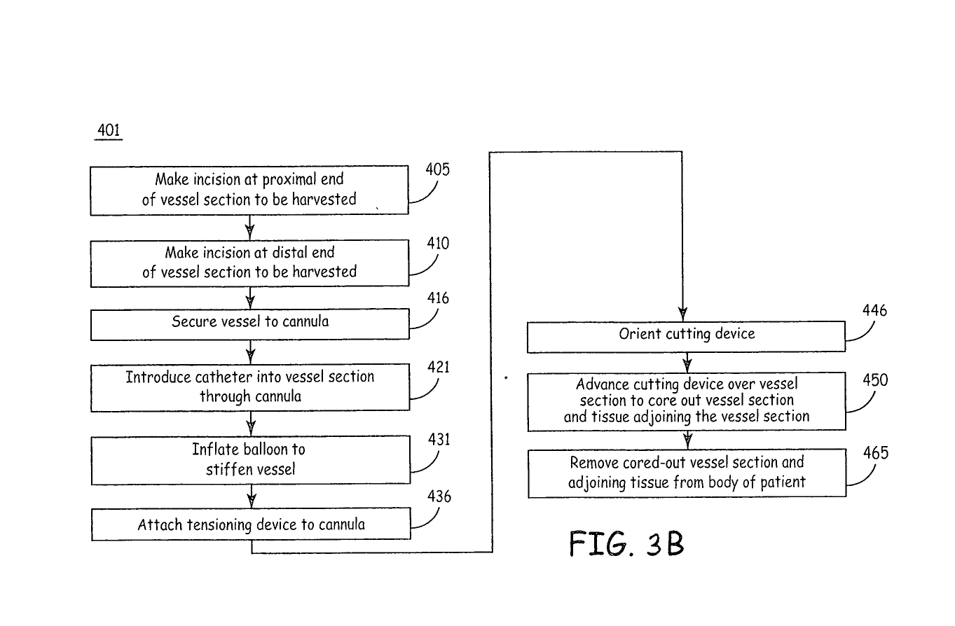

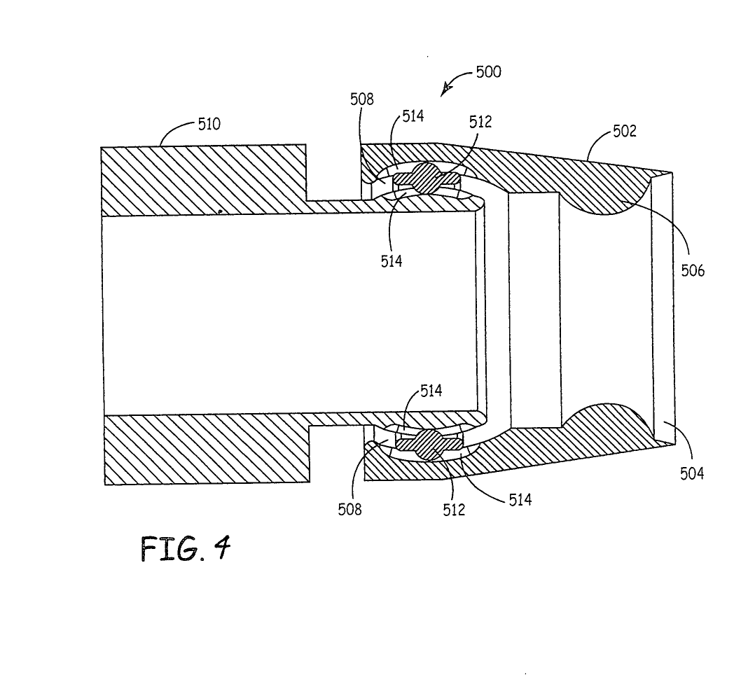

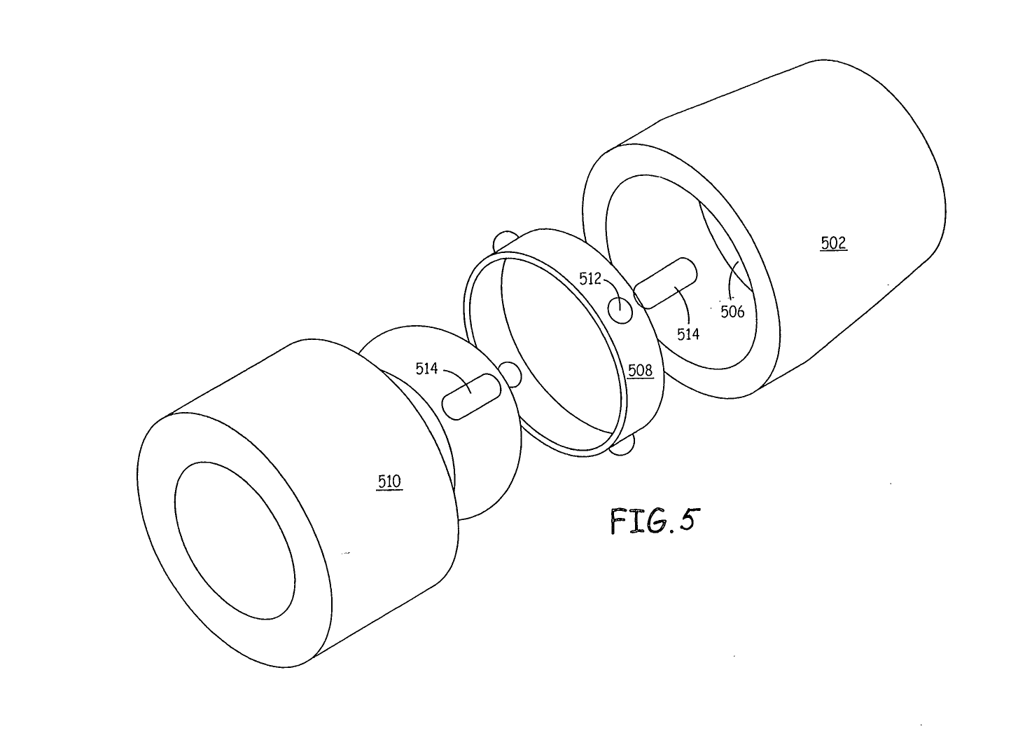

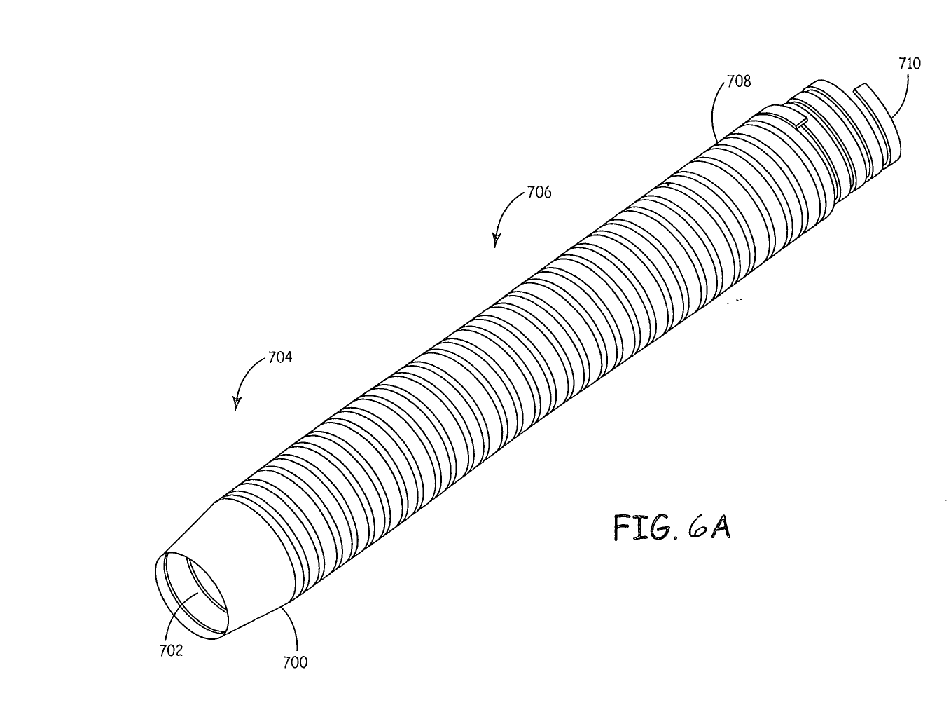

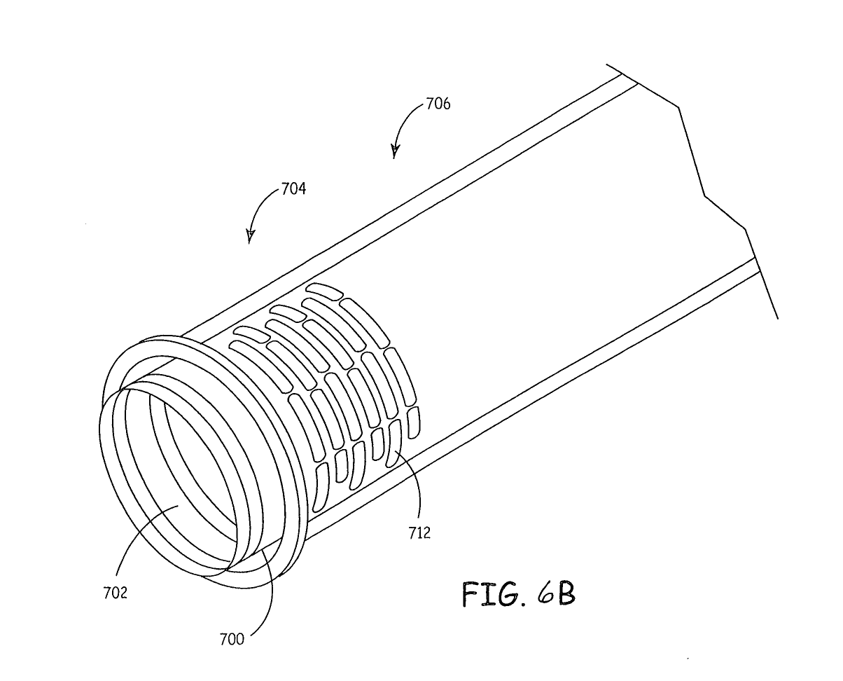

FIG. 3A is a flow diagram illustrating a vessel harvesting method according to one embodiment of the invention. In this embodiment, a first incision is made at a point corresponding to a proximal end of the vessel section to be harvested (Block 405). A second incision is made at a point corresponding to a distal end of the vessel section (Block 410). A guidewire can then be positioned within the vessel section (Block 415). Alternatively, the guidewire can be inserted into the vessel before the second incision is made. Inserting the guidewire before making the second incision can aid in determining the optimal location for the second incision. Once the second incision has been made, the guidewire can be positioned such that it extends beyond and outside of the vessel section at both the distal and proximal ends of the section.

A catheter can be introduced into the vessel section over the previously placed guidewire (Block 420). A proximal portion of the vessel section can be secured to the catheter (Block 425), for example by suturing the vessel onto a barb positioned adjacent to the proximal end of the catheter. Alternatively, the catheter can be introduced into the vessel without a guidewire being previously placed.

The guidewire (if present) is withdrawn (Block 430), and a rod can be inserted into the catheter to stiffen the vessel section (Block 435). Both the catheter and the rod can be attached to a removable handle (Block 440). The handle can carry the cutting device 100, or the cutting device 100 can be introduced over the handle after the handle has been attached to the catheter and rod. An inner lumen of the cutting device 100 can provide a close-sliding fit for the handle. The cutting device 100 can thus be oriented coaxial with the rod and with the vessel section to be harvested (Block 445).

The cutting device 100 is then advanced over the vessel section to core out the vessel section and tissue adjoining the vessel section (Block 450). The cutting device 100 can be advanced by either pushing or pulling the device over the vessel section. If the cutting device 100 includes two tubular members 110, 120, one positioned within the other as shown in FIG. 1, the two tubular members 110, 120 can be advanced separately. For example, the inner tubular member 120 can be advanced first to hold the vessel and surrounding tissue, while the outer tubular member 110 is advanced second to cut the tissue being held by the inner tubular member 120. The process of incrementally advancing the inner tubular member 120 and then the outer tubular member 110 can be repeated until the entire section has been excised. In one embodiment, advancing the inner tubular member 120 ahead of the outer tubular member 110 can protect the walls of the vessel from the cutting element 130 positioned on the outer tubular member 110. Advancing and rotating the inner and outer tubular members 110, 120 separately can also protect the side branches of the vessel by holding them in place to achieve a clean cut at a sufficient length. The cutting device 100, for example, can be twisted first in one direction and then in the other direction, or it can be rotated over the vessel. The outer and inner tubular members 110, 120 can be twisted in opposite directions to provide a scissoring action.

The cored out vessel section and adjoining tissue are removed from the body of the patient (Block 455). Either before or after removing the vessel section and adjoining tissue, a hemostatic control method for branch vessels severed as a result of coring out the vessel section can be introduced through either the first or the second incision. The hemostatic control method can be, for example, a biological sealant, e.g., platelet gel that can be prepared from the patient's blood and injected or otherwise introduced along the track of the cutting device. The hemostatic control method can also be a thrombogenic substance such as fibrinogen, fibrin and/or thrombin placed in the track left by the cutting device 100. Alternatively, or in combination with a biological sealant, a biocompatible or biodegradable tube can be enclosed within the cutting device 100 to be delivered as the cutting device 100 is advanced over the vessel or after the cutting device 100 has completed coring out the vessel and adjoining tissue. The tube exerts pressure on the cut branch vessels and can be either removed or, in the case of a biodegradable tube, left to dissolve or degrade over a period of a few days, for example. The space left after the removal of the vessel can also be filled with gauze to provide internal pressure to limit bleeding and absorb blood. The gauze can be removed periodically to check for absorbed blood. Limited blood collected on the gauze indicates the wound bleeding has diminished.

Hemostatic control methods are not required for embodiments of the invention as the tubular cutting device 100 itself can exert pressure on the cut branch vessels while it remains within the patient's body. A drain can be inserted at the end of the harvesting procedure to deal with any bleeding that does occur. The site of the vessel harvesting procedure, e.g., the leg of a patient, can also be wrapped with a compression bandage to limit bleeding.

FIG. 3B is a flow diagram illustrating a vessel harvesting method according to another embodiment of the invention. A first incision is made at a point corresponding to a proximal end of the vessel section to be harvested (Block 405). A second incision is made at a point corresponding to a distal end of the vessel section (Block 410). A cannula is then inserted into the proximal end of the vessel section, which is located near the knee. The proximal end of the vessel is then secured to the cannula (Block 416), for example by suturing the vessel onto a barb or raised portion positioned adjacent to the distal end of the cannula. A balloon catheter is then introduced through the cannula and positioned within the vessel section (Block 421). Once positioned, the balloon is inflated to stiffen the vessel section (Block 431). A vessel-tensioning device or system is then attached to the cannula to provide a vessel-tensioning force to the vessel section (Block 436).

The cutting device 100 is oriented coaxially with the cannula, the balloon and the vessel section to be harvested (Block 446). The cutting device 100 is then advanced over the vessel section to core out the vessel section and tissue adjoining the vessel section (Block 450). The cutting device 100, for example, can be twisted first in one direction and then in the other direction, or it can be rotated over the vessel. The cored out vessel section and adjoining tissue are removed from the body of the patient (Block 455). Either before or after removing the vessel section and adjoining tissue, a hemostatic control method for treating branch vessels severed as a result of coring out the vessel section can be introduced through either the first or the second incision. The hemostatic control method can include, for example, a biological sealant, e.g., platelet gel that can be prepared from the patient's blood and injected or otherwise introduced along the track of the cutting device 100. The hemostatic control method can also be a thrombogenic substance such as fibrinogen, fibrin and/or thrombin placed in the track left by the cutting device 100. Alternatively, or in combination with a biological sealant, a biocompatible or biodegradable tube can be enclosed within the cutting device 100 to be delivered as the cutting device 100 is advanced over the vessel or after the cutting device 100 has completed coring out the vessel and adjoining tissue. The tube exerts pressure on the cut branch vessels and can be either removed or, in the case of a biodegradable tube, left to dissolve or degrade over a period of a few days, for example. The space left after the removal of the vessel can also be filled with gauze to provide internal pressure to limit bleeding and absorb blood. The gauze can be removed periodically to check for absorbed blood. Limited blood collected on the gauze indicates the wound bleeding has diminished.

FIG. 4 illustrates a distal end 500 of a cutting device 502 for harvesting a vessel section according to one embodiment of the invention. The distal end 500 of the cutting device 502 is comprised of a cutting element 504, a routing ridge or ring-like structure 506, a connector section 508, and a tubular member 510. As shown, the tubular member 510 is operably coupled to the cutting device 502 via the connector section 508. The connector section 508 has rounded surface features 512, which fit within grooves 514 and hold the tubular member 510 to the cutting device 502 (as also shown in FIG. 5). The relationship of the grooves 514 and the surface features 512 allows the cutting device 502 to tip or cant off axis from the tubular member 510, but still allows the cutting device 502 to rotate as the tubular member 510 is rotated.

Regardless of the device being used to stabilize or support the vessel, the routing ridge 506 can contact the tissue being cut and meets resistance against the stabilizing or support device inside the vessel. The contact and resistance exerted against the routing ridge 506 pushes on the cutting device 502 to center the cutting device 502 around the vessel. Thus, as the tubular member 510 is advanced over the vessel, if the vessel is curved in any way, the routing ridge 506 will contact the vessel first before the cutting element 504, thereby centering the cutting element 504 over the vessel prior to the cutting element 504 coming into contact with the vessel and thus avoiding any damage to the vessel.

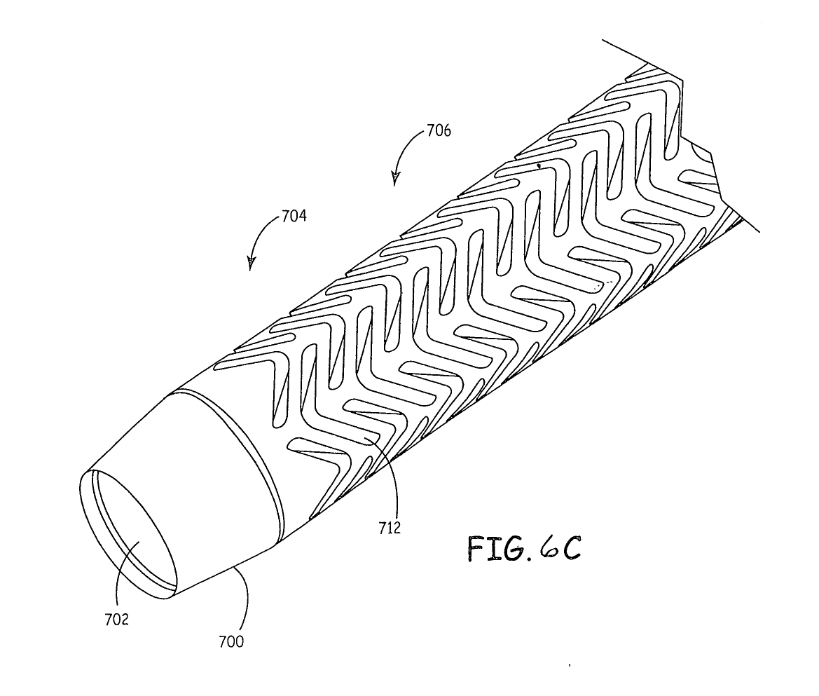

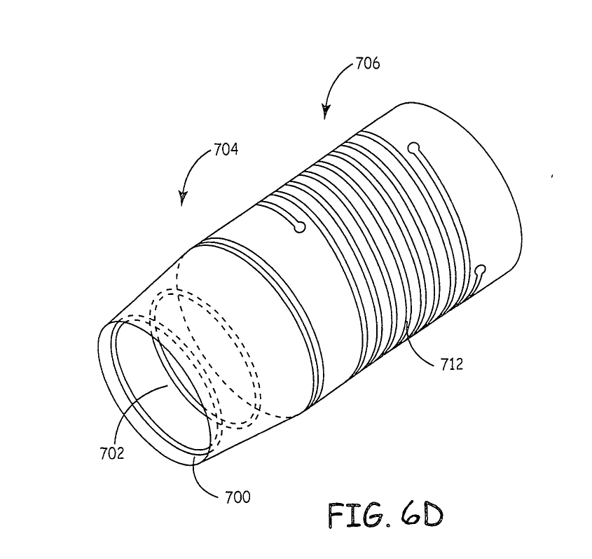

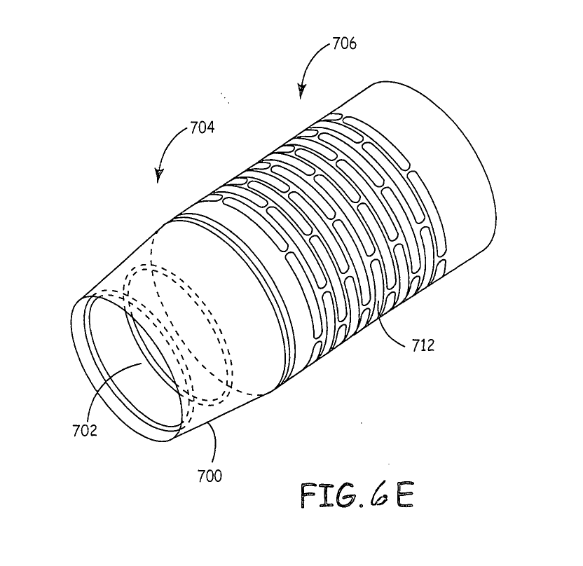

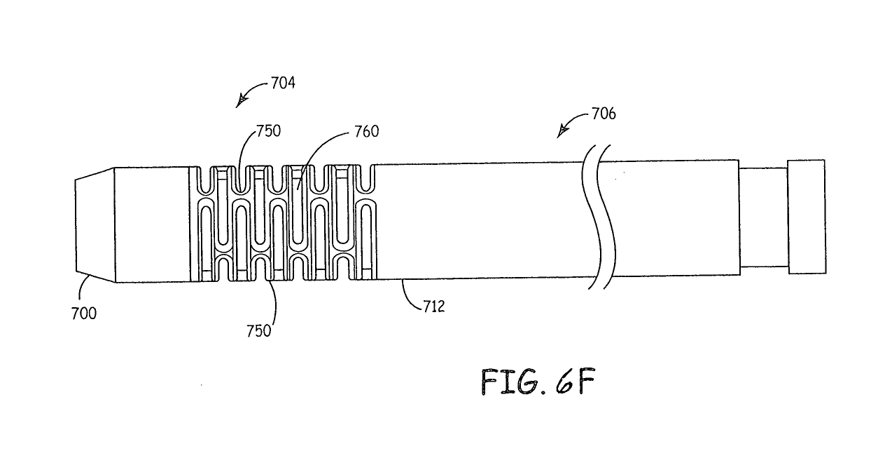

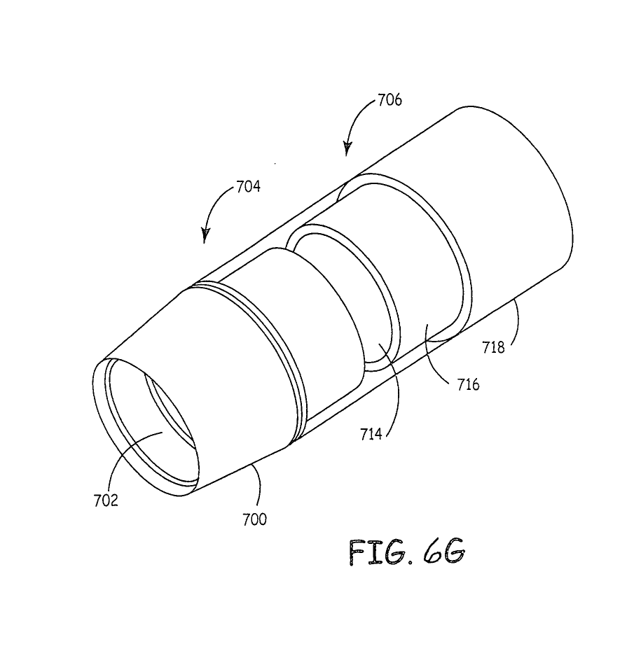

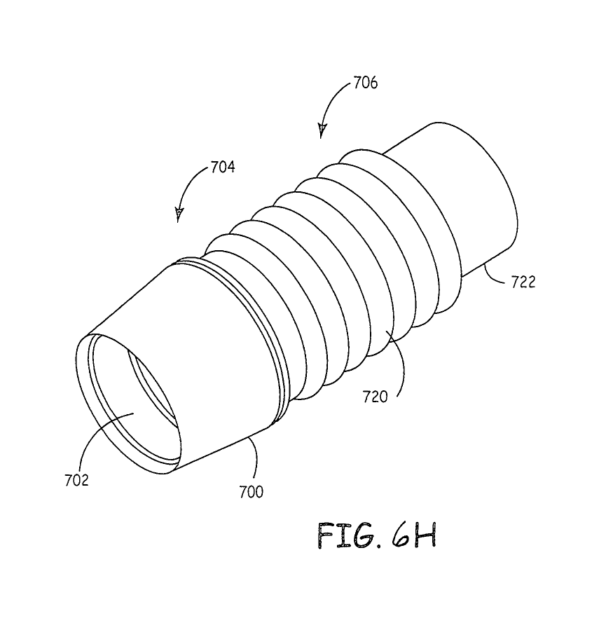

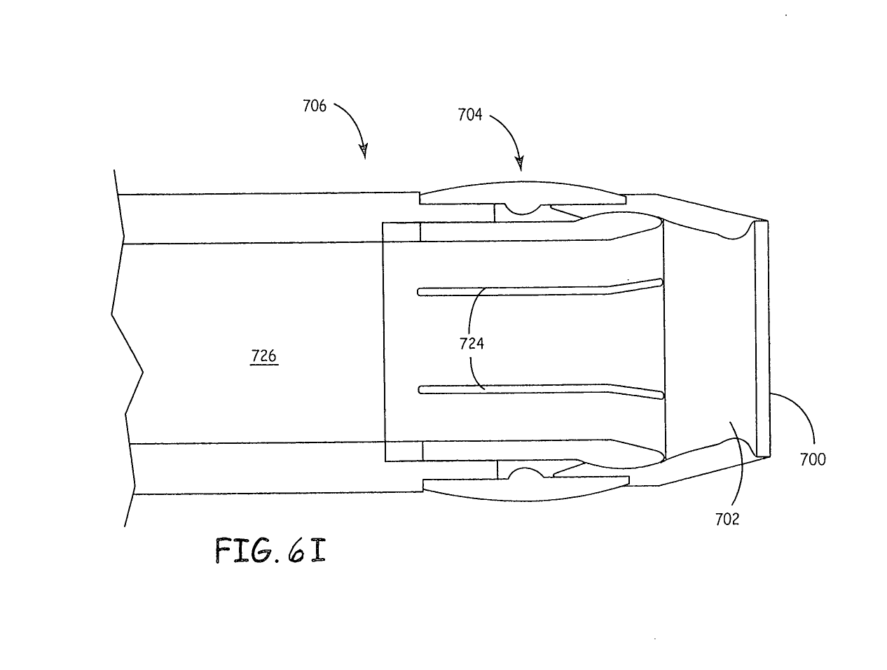

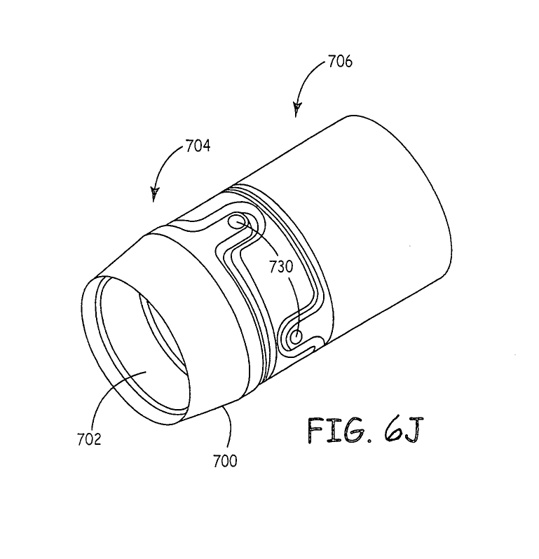

FIGS. 6A-6J illustrate distal ends 704 of cutting devices 706 according to various embodiments of the invention. In these embodiments, a cutting element 700 and a routing ridge 702 at the distal end 704 are similar in structure to the embodiments of FIGS. 4 and 5. However, the embodiments of FIGS. 6A-6J provide a flexible distal end 704 to assist the cutting device 706 in navigating over a vessel during harvesting. Each flexible distal end 704 has a construction that allows the distal end 704 to bend or flex and thus more easily navigate around curvatures of the vessel. The flexible distal end 704 provides increased tracking response and flexibility.

The embodiment of FIG. 6A includes a dual-coiled structure having a counterclockwise-wound outer coil 708 and a clockwise-wound inner coil 710. The dual-coil structure provides for a torsionally-stable tube strong enough to withstand external compression, yet still remain flexible due to the coil nature.

FIGS. 6B-6F illustrate various perforated tube sections. These perforations or slots weaken the tubing wall just enough to allow the tubing to be flexible enough to route the cutting device 706 around the vessel, especially curved vessels. The perforated tubing 712 can be made from a biocompatible metal, such as stainless steel, or a biocompatible plastic, such as polyurethane, acrylic, polyvinylchloride (PVC) and/or a similar material. This perforated geometry can provide good flexibility and high torque. The portions in the perforated sections that run coaxially with the center of the tubular member, i.e., the posts, can facilitate providing torque.

As shown in FIG. 6F, posts 750, beams 755, and perforated slots 760 increase in size from the distal portion to the proximal portion. This variation in size stimulates a tapered beam, e.g., a fishing pole, to allow for the greatest flexibility at the distal end while distributing the stress along its length.

As shown in FIG. 6G, multiple tubes 714, 716, and 718 are used to provide the flexible distal end 704. Each tube 714, 716, and 718 can terminate at a different proximal distance than the other. Each tube 714, 716, and 718 can be made of a biocompatable material, such as ePTFE (expanded polytetrafluoroethylene), silicone, polyvinylchloride and/or similar material, which is very flexible and keeps its shape when compressed and extended axially. The tube 714 extends to the cutting element 700, the tube 716 terminates a distance proximal of the cutting element 700, and the tube 718 terminates a further distance proximal to the cutting element 700. By wrapping the tubes 714, 716, and 718 around one another, the amount of torque that can be applied to the cutting device 706 can be increased. As the tube diameter decreases towards the distal end 704, the distal end 704 becomes more flexible and thus able to navigate over vessels having small to large curvature.

FIG. 6H illustrates a flex straw portion 720 that can be constructed from the same material as the tubing 722, provided that the material is biocompatible and has good flexing capabilities. The flex straw portion 720 provides a good torque quotient in that the flex straw portion 720 can be compressed to solid, but yet provides for good flexibility when not compressed. FIG. 61 illustrates an embodiment where perforations 724 run lengthwise along tubing member 726. FIG. 6J illustrates a cutting device 706 having multiple universal joints 730 to provide for a flexible distal end 704. One universal joint 730 provides for flexibility in a vertical plane, while another universal joint 730 provides for flexibility in a horizontal plane. In other embodiments, the distal end 704 can have other designs providing distal end flexibility. The distal end 704 can be manufactured from a suitable biocompatible material including metals, ceramics, and plastics.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

Various features and advantages of the invention are set forth in the following claims.

1. A cutting device for use in harvesting a section of a vessel from surrounding tissue, the cutting device comprising:
at least one tubular member adapted to surround the vessel along the section of the vessel to be harvested;
a cutting element coupled to the at least one tubular member, the cutting element adapted to be moved along the section of the vessel in order to cut the tissue around the vessel; and
a centering member coupled to one of the at least one tubular member and the cutting element, the centering member adapted to keep the vessel spaced from the cutting element as the cutting element cuts the tissue around the vessel.

2. The cutting device of claim 1 wherein the at least one tubular member includes a single outer tubular member with the cutting element attached to a distal end of the single outer tubular member.

3. The cutting device of claim 1 wherein the at least one tubular member is constructed of at least one of a biocompatible polymer and stainless steel.

4. The cutting device of claim 1 wherein a distal end of the at least one tubular member is flexible.

5. The cutting device of claim 1 wherein the at least one tubular member has a length substantially equal to a length of the section of the vessel to be harvested.

6. The cutting device of claim 1 wherein at least a portion of the cutting device is coated with a lubricious coating.

7. The cutting device of claim 1 wherein the cutting element is mounted on an outside portion of the at least one tubular member.

8. The cutting device of claim I wherein the cutting element includes at least one of a blade having a taper on an outside surface, a blade having a taper on an inside surface, a ring having a sharpened edge, and a ring having a beveled edge.

9. The cutting device of claim 1 wherein the cutting element includes a blade having a blunt edge on a first surface and a sharp edge on a second surface, and wherein the blunt edge provides a buffer space between the vessel and the sharp edge.

10. The cutting device of claim I wherein when the cutting element begins to deviate from a centered position, the centering member places a force on at least one of the vessel, the tissue surrounding the vessel, and the centering member, resulting in the cutting device being directed back to the centered position.

11. The cutting device of claim I wherein the at least one tubular member is advanced over the section of the vessel to be harvested by at least one of pushing, pulling, rotating, and twisting.

12. A method of harvesting a section of a vessel from surrounding tissue, the method comprising:
making a first incision at a proximal end of the section of the vessel;
making a second incision at a distal end of the section of the vessel;
introducing a vessel support device into the vessel;
orienting a cutting device coaxially with the vessel support device;
advancing the cutting device over the vessel to core out the section of the vessel and a portion of the surrounding tissue; and
spacing a cutting element of the cutting device from the vessel as the cutting element is advanced over the vessel.

13. The method of claim 12 wherein spacing the cutting element of the cutting device from the vessel includes substantially centering the vessel within the cutting device as the cutting element is advanced over the vessel.

14. The method of claim 12 wherein the vessel support device includes a catheter, and further comprising introducing the catheter into the vessel and advancing the cutting device over the catheter and the vessel.

15. The method of claim 12 and further comprising incrementally advancing the cutting device over the section of the vessel by at least one of pulling, pushing, rotating, and twisting the cutting device.

16. The method of claim 12 and further comprising providing hemostatic control for branches severed from the section of the vessel.

17. The method of claim 12 and further comprising inserting a cannula into the proximal end of the section of the vessel and securing the proximal end of the section of the vessel to the cannula.

18. The method of claim 17 and further comprising introducing a vessel support device including a balloon catheter through the cannula and into the section of the vessel.

19. The method of claim 18 and further comprising attaching the cannula to a vessel-tensioning device.

20. A cutting device for use in harvesting a section of a vessel from surrounding tissue, the cutting device comprising:
at least one tubular member adapted to surround the vessel along the section of the vessel to be harvested, the at least one tubular member including a flexible section on a portion of a distal end; and
a cutting element coupled adjacent to the flexible section of the at least one tubular member, the cutting element adapted to be moved along the section of the vessel in order to cut the surrounding tissue.

21. The cutting device of claim 20 wherein the flexible section includes a perforated portion of the at least one tubular member.

22. The cutting device of claim 21 wherein the perforated portion includes slots that increase in size from a distal portion to a proximal portion of the flexible section.

23. The cutting device of claim 20 wherein the at least one tubular member includes a single outer tubular member with the cutting element attached to a distal end of the single outer tubular member.

24. The cutting device of claim 20 wherein the cutting element is mounted on an outside portion of the at least one tubular member adjacent to the flexible section.

25. The cutting device of claim 20 wherein the cutting element includes at least one of a blade having a blunt edge on a first surface and a sharp edge on a second surface, a blade having a taper on an outside surface, a blade having a taper on an inside surface, a ring having a sharpened edge, and a ring having a beveled edge.
